(19) United States
(12) Patent Application Publication
(10) Pub. No.: US 2022/0151901 A1
(43) Pub. Date: May 19, 2022

(54) A METHOD OF COLORING KERATINOUS MATERIAL COMPRISING THE USE OF AN ORGANOSILICON COMPOUND, A COLORING COMPOUND, AND A MIXTURE OF SILICONES

(71) Applicant: Henkel AG & Co. KGaA, Duesseldorf (DE)

(72) Inventors: Rene KROHN, Norderstedt (DE); Thomas HIPPE, Appen (DE); Stefan HOEPFNER, Hamburg (DE); Jessica BRENDER, Hamburg (DE)

(73) Assignee: Henkel AG & Co. KGaA, Duesseldorf (DE)

(21) Appl. No.: 17/438,887

(22) PCT Filed: Feb. 2, 2020
(86) PCT No.: PCT/EP2020/055079

§ 371 Date: Sep. 13, 2021

(30) Foreign Application Priority Data
Mar. 12, 2019 (DE) 10 2019 203 298.2

(51) Int. Cl. A61K 8/58; A61K 8/892; A61K 8/898; A61K 8/36; A61K 8/365; A61K 8/44; A61K 8/92; A61K 8/34; A61Q 5/10

(52) U.S. Cl. CPC A61K 8/585; A61K 8/892; A61K 8/898; A61K 8/361; A61K 8/365; A61K 2800/594; A61K 8/92; A61K 8/342; A61Q 5/10; A61K 2800/4324; A61K 2800/884; A61K 8/44

(57) ABSTRACT

The subject of the present disclosure is a process for dyeing keratinous material, in particular human hair, comprising the following steps:
Application of an agent (a) to the keratinous material, wherein the agent (a) comprises at least one organic silicon compound,
Application of an agent (b) to the keratinous material, the agent (b) comprising at least one colorant compound including comprising selected from the group of pigments and/or direct dyes,
Application of an agent (c) to the keratinous material, wherein the agent (c) comprises:
   (c1) a hydroxy-terminated polyorganosiloxane, and
   (c2) a reaction product of a hydroxy terminated polyorganosiloxane with an acid and/or an alcohol and/or a wax.

### CROSS-REFERENCE TO RELATED APPLICATION

This application is a U.S. National-Stage entry under 35 U.S.C. § 371 based on International Application No. PCT/EP2020/055079, filed Feb. 27, 2020, which was published under PCT Article 21(2) and which claims priority to German Application No. 102019203298.2, filed Mar. 12, 2019, which are all hereby incorporated in their entirety by reference.

### TECHNICAL FIELD

The subject of the present application is a process for dyeing keratinous material, in particular human hair, which comprises the application of three different agents (a), (b) and (c). The agent (a) comprises at least one organic silicon compound. The agent (b) comprises at least one colorant compound including comprising selected from the group of pigments and/or direct dyes. Characteristic of agent (c) is a mixture of selected silicones.

A second subject of this application is a multi-component packaging unit (kit-of-parts) for dyeing keratinous material, in particular human hair, which comprises means (a), (b) and (c) separately assembled in three different containers.

### BACKGROUND

Changing the shape and color of keratinous material, especially human hair, is an important area of modern cosmetics. To change the hair color, the expert knows various coloring systems depending on the coloring requirements. Oxidation dyes are usually used for permanent, intensive dyeing's with good fastness properties and good grey coverage. Such colorants contain oxidation dye precursors, so-called developer components and coupler components, which, under the influence of oxidizing agents such as hydrogen peroxide, form the actual dyes among themselves. Oxidation dyes are characterized by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents comprising surfactants. Various products of this type are available on the market under the name hair mascara.

If the user desires a particularly long-lasting coloring of his hair, the use of oxidative dyes is his only option so far. However, despite numerous optimization attempts, an unpleasant ammonia or amine odor cannot be completely avoided in oxidative hair dyeing. The hair damage still associated with the use of oxidative dyes also has a negative effect on the user's hair. A continuing challenge is therefore the search for alternative, high-performance dyeing processes.

EP 2168633 B1 deals with the task of producing long-lasting hair colorations using pigments. The paper teaches that when a combination of pigment, organic silicon compound, hydrophobic polymer and a solvent is used on hair, it is possible to create colorations that are said to be particularly resistant to shampooing.

In WO 2018/115059 A1 a dyeing process is described, which runs in several steps. One step involves the application of an organ silane, and another step involves the application of a direct dye to the hair. This process is also used to achieve dyeing's with good wash fastness properties.

However, there is still a need to improve the wash fastness of dyeing's based on pigments and/or direct dyes and without oxidation dye precursors.

### BRIEF SUMMARY

***the attorney will review the specification and include their own brief summary***

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Accordingly, the task of the present disclosure was to provide a dyeing system that has fastness properties comparable to oxidative dyeing. Wash fastness properties should be outstanding, but the use of oxidation dye precursors normally used for this purpose should be avoided. A technology was sought that would make it possible to fix colorant compounds known from the prior art (such as pigments or direct-acting dyes) to the hair in an extremely durable manner.

Surprisingly, it has now been found that the task can be excellently solved if keratinous materials, in particular human hair, are colored by a process in which at least three agents (a), (b) and (c) are applied to the keratinous materials (hair). Here, agent (a) comprises at least one organic silicon compound, agent (b) comprises at least one pigment and/or direct-acting dye, and agent (c) comprises a mixture of selected hydroxy-terminated silicones.

When the three agents (a), (b) and (c) were used in a dyeing process, keratinous material could be dyed with particularly high color intensity.

A first object of the present disclosure is a method for coloring keratinous material, in particular human hair, comprising the following steps:

Application of an agent (a) to the keratinous material, wherein the agent (a) comprises at least one organic silicon compound,

Application of an agent (b) to the keratinous material, the agent (b) comprising at least one colorant compound including comprising selected from the group of pigments and/or direct dyes,

Application of an agent (c) to the keratinous material, wherein the agent (c) comprises:

(c1) a hydroxy terminated polyorganosiloxane, and
(c2) a reaction product of a hydroxy terminated polyorganosiloxane with an acid and/or an alcohol and/or a wax.

Keratinous Material

Keratinous material includes hair, skin, nails (such as fingernails and/or toenails). Wool, furs, and feathers also fall under the definition of keratinous material.

Preferably, keratinous material is understood to be human hair, human skin, and human nails, especially fingernails and toenails. Keratinous material is understood to be human hair.

Agent (a), (b) and (c)

In the process as contemplated herein, agents (a), (b) and (c) are applied to the keratinous material, in particular human hair. The three agents (a), (b) and (c) are different from each other.

Accordingly, a method for dyeing keratinous material, in particular human hair, is disclosed, comprising the following steps:

Application of an agent (a) to the keratinous material, wherein the agent (a) comprises at least one organic silicon compound,

Application of an agent (b) to the keratinous material, the agent (b) comprising at least one colorant compound including comprising selected from the group of pigments and/or direct dyes, and

Application of an agent (c) to the keratinous material, wherein the agent (c) comprises:

(c1) a hydroxy terminated polyorganosiloxane, and
(c2) a reaction product of a hydroxy terminated polyorganosiloxane with an acid and/or an alcohol and/or a wax,

where the three agents (a), (b) and (c) are different from each other.

Agent (a)

Agent (a) is characterized by its content of at least one organic silicon compound, in particular at least one organic silane. The organic silicon compounds or organic silanes included in agent (a) is reactive compounds.

Composition (a) comprises the organic silicon compound(s), in particular the organic silane(s), in a cosmetic carrier which may be hydrated, low in water or anhydrous. In addition, the cosmetic carrier can be liquid, gel-like, creamy, pasty, powdery or even solid (e.g., in the form of a tablet or a pressed product). Preferably, the cosmetic carrier of the product (a) is an aqueous or aqueous-alcoholic carrier. To hair coloring, such carriers are, for example, creams, emulsions, gels, or also surfactant-comprising foaming solutions, such as shampoos, foam aerosols, foam formulations or other preparations suitable for application to the hair.

The cosmetic carrier preferably comprises water, which means that the carrier comprises at least 2% by weight of water based on its weight. Preferably, the water content is above 5 wt. %, further preferably above 10 wt. % still further preferably above 15 wt. %. The cosmetic carrier can also be aqueous alcoholic. [0206] Aqueous/alcoholic solutions in the context of the invention are aqueous solutions comprising 2 to 70% by weight of a C1-C4 alcohol, more particularly ethanol or isopropanol. The agents may additionally contain other organic solvents, such as methoxy butanol, benzyl alcohol, ethyl Di glycol or 1,2-propylene glycol. Preferred are all water-soluble organic solvents.

The term “coloring agent” is used in the context of the present disclosure to refer to a coloring of keratinous material, in particular human hair, brought about using pigments and/or direct dyes. During this coloring process, the coloring compounds are deposited in a particularly homogeneous and smooth film on the surface of the keratinous material or diffuse into the keratinous fiber. The film is formed in situ by oligomerization or polymerization of the organic silicon compound(s), and by the interaction of organic silicon compound with the colorant compounds.

Organic Silicon Compounds

As an essential ingredient of the invention, the agent (a) comprises at least one organic silicon compound. Preferred organic silicon compounds are selected from silanes having one, two or three silicon atoms.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds of the invention are preferably compounds comprising one to three silicon atoms. Organic silicon compounds preferably contain one or two silicon atoms.

The agent (a) particularly preferably comprises at least one organic silicon compound selected from silanes having one, two or three silicon atoms.

According to IUPAC rules, the term silane chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

In a particularly preferred embodiment, the method is characterized by the application of an agent (a) to the keratinous material, wherein the agent (a) comprises at least one organic silicon compound selected from silanes having one, two or three silicon atoms.

The agent (a) particularly preferably comprises at least one organic silicon compound selected from silanes having one, two or three silicon atoms, the organic silicon compound further comprising one or more basic chemical functions and one or more hydroxyl groups or hydrolysable groups per molecule.

In a very particularly preferred embodiment, the method is characterized by the application of an agent (a) to the keratinous material, said agent (a) comprising at least one organic silicon compound selected from silanes having one, two or three silicon atoms, said organic silicon compound further comprising one or more basic chemical functions and one or more hydroxyl groups or hydrolysable groups per molecule.

This basic group can be, for example, an amino group, an alkylamino group or a dialkylamino group, which is preferably connected to a silicon atom via a linker. Preferably, the basic group is an amino group, a C1-C6 alkylamino group or a di(C1-C6)alkylamino group.

The hydrolysable group(s) is (are) preferably a C1-C6 alkoxy group, especially an ethoxy group or a methoxy group. It is preferred when the hydrolysable group is directly bonded to the silicon atom. For example, if the hydrolysable group is an ethoxy group, the organic silicon compound preferably comprises a structural unit R′R″R″′Si—O—CH2-CH3. The radicals R′, R″ and R′″ represent the three remaining free valences of the silicon atom.

A very particularly preferred method is wherein the agent (a) comprises at least one organic silicon compound selected from silanes having one, two or three silicon atoms, the organic silicon compound preferably comprising one or more basic chemical functions and one or more hydroxyl groups or hydrolysable groups per molecule.

Particularly good results were obtained when the agent (a) comprises at least one organic silicon compound of formula (I) and/or (II).

In another very particularly preferred embodiment, a method is wherein an agent (a) is applied to the keratinous material or human hair, wherein the agent (a) comprises at least one organic silicon compound (a) of formula (I) and/or (II),

R1R2N-L-Si(OR3)a(R4)b (I),

where
R1, R2 independently represent a hydrogen atom or a C1-C6 alkyl group,
L is a linear or branched divalent C1-C20 alkylene group,
R3 represents a hydrogen atom or a C1-C6 alkyl group,
R4 represents a C1-C6 alkyl group
a, stands for an integer from 1 to 3, and
b stands for the integer 3-a,

(R5O)c(R6)dSi-(A)e—[NR7-(A′)]f-[O-(A″)]g—[NR8-(A′″)]h-Si(R6′)d′(OR5′)c′ (II),

where
R5, R5′, R5″ independently represent a hydrogen atom or a C1-C6 alkyl group,
R6, R6′ and R6″ independently represent a C1-C6 alkyl group,
A, A′, A″, independently of one another represent a linear or branched divalent C1-C20 alkylene group
R7 and R8 independently represent a hydrogen atom, a C1-C6 alkyl group, a hydroxy C1-C6 alkyl group, a C2-C6 alkenyl group, an amino C1-C6 alkyl group or a group of formula (III)

(A″″)-Si(R6″)d″(OR5″)c″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

The substituents R1, R2, R3, R4, R5, R5′, R5″, R6, R6′, R6″, R7, R8, L, A, A′, A″, A′″ and A″″ in the compounds of formula (I) and (II) are explained below as examples:

Examples of a C1-C6 alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C2-C6 alkenyl group are vinyl, allyl, but-2-enyl, but-3-enyl and isobutenyl, preferred C2-C6 alkenyl radicals are vinyl and allyl. Preferred examples of a hydroxy C1-C6 alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C1-C6 alkyl group are the aminomethyl group, the 2-aminoethyl group, the 3-aminopropyl group. The 2-aminoethyl group is particularly preferred. Examples of a linear divalent C1-C20 alkylene group include the methylene group (—CH2),), the ethylene group (—CH2-CH2-), the propylene group (—CH2-CH2-CH2-) and the butylene group (—CH2-CH2-CH2-CH2-). The propylene group (—CH2-CH2-CH2-) is particularly preferred. From a chain length of 3 C atoms, divalent alkylene groups can also be branched. Examples of branched divalent C3-C20 alkylene groups are (—CH2-CH(CH3)-) and (—CH2-CH(CH3)-CH2-).

In the organic silicon compounds of the formula (I)

R1R2N-L-Si(OR3)a(R4)b (I),

the radicals R1 and R2 independently of one another represent a hydrogen atom or a C1-C6 alkyl group. In particular, the radicals R1 and R2 both represent a hydrogen atom.

In the middle part of the organic silicon compound is the structural unit or the linker -L- which stands for a linear or branched, divalent C1-C20 alkylene group.

Preferably -L- stands for a linear, divalent C1-C20 alkylene group. Further preferably -L- stands for a linear divalent C1-C6 alkylene group. Particularly preferred -L stands for a methylene group (CH2-), an ethylene group (—CH2-CH2-), propylene group (—CH2-CH2-CH2-) or butylene (—CH2-CH2-CH2-CH2-). L stands for a propylene group (—CH2-CH2-CH2-)

The organic silicon compounds of formula (I)

R1R2N-L-Si(OR3)a(R4)b (I),

one end of each carries the silicon-comprising group —Si(OR3)a(R4)b

In the terminal structural unit —Si(OR3)a(R4)b, R3 is hydrogen or C1-C6 alkyl group, and R4 is C1-C6 alkyl group. Particularly preferred, R3 and R4 independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Dyeing's with the best wash fastnesses could be obtained if the agent (a) comprises at least one organic silicon compound of formula (I) in which the radicals R3, R4 independently of one another represent a methyl group or an ethyl group.

Furthermore, dyeing's with the best wash fastnesses could be obtained if the agent (a) comprises at least one organic silicon compound of the formula (I) in which the radical a represents the number 3. In this case the rest b stands for the number 0.

In another preferred embodiment, an agent (a) is wherein it comprises at least one organic silicon compound of formula (I),

where
R3, R4 independently of one another represent a methyl group or an ethyl group and
a stands for the number 3 and
b stands for the number 0.

In another preferred embodiment, the method is wherein the agent (a) comprises at least one organic silicon compound of formula (I),

R1R2N-L-Si(OR3)a(R4)b (I),

where
R1, R2 both represent a hydrogen atom, and
L represents a linear, divalent C1-C6-alkylene group, preferably a propylene group (—CH2-CH2-CH2-) or an ethylene group (—CH2-CH2-),
R3 represents a hydrogen atom, an ethyl group, or a methyl group,
R4 represents a methyl group or an ethyl group,
a stands for the number 3 and
b stands for the number 0.

When b is 0, the radical R4 does not occur in the compounds of formula (I).

Accordingly, in a further preferred embodiment, the method is wherein the agent (a) comprises at least one organic silicon compound of formula (I),

R1R2N-L-Si(OR3)a(R4)b (I),

where
R1, R2 both represent a hydrogen atom, and
L represents a linear, divalent C1-C6-alkylene group, preferably a propylene group (—CH2-CH2-CH2-) or an ethylene group (—CH2-CH2-),
R3 represents a hydrogen atom, an ethyl group, or a methyl group,
a stands for the number 3 and
b stands for the number 0.

Organic silicon compounds of the formula (I) which are particularly suitable for solving the problem are

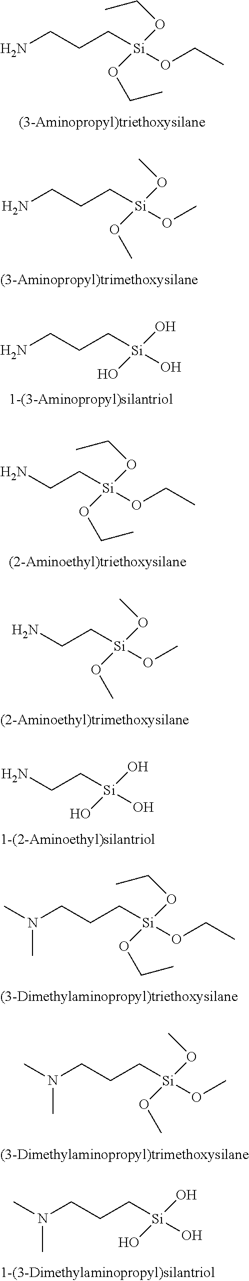

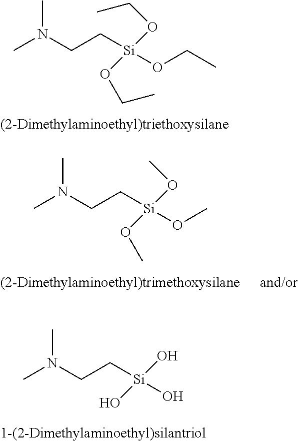

In a further preferred embodiment, the method is wherein the agent (a) comprises at least one organic silicon compound of formula (I) including comprising selected from the group of
(3-Aminopropyl)triethoxysilane
(3-Aminopropyl)trimethoxysilane
1-(3-Aminopropyl)silantriol
(2-Aminoethyl)triethoxysilane
(2-Aminoethyl)trimethoxysilane
1-(2-Aminoethyl)silantriol
(3-Dimethylaminopropyl)triethoxysilane
(3-Dimethylaminopropyl)trimethoxysilane
1-(3-Dimethylaminopropyl)silantriol
(2-Dimethylaminoethyl)triethoxysilane
(2-Dimethylaminoethyl)trimethoxysilane,
1-(2-Dimethylaminoethyl)silantriol
   and mixtures thereof.

The organic silicon compound of formula (I) is commercially available.

(3-aminopropyl)trimethoxysilane, for example, can be purchased from Sigma-Aldrich. (3-aminopropyl)triethoxysilane is also commercially available from Sigma-Aldrich.

In a further embodiment, the agent (a) comprises at least one organic silicon compound of formula (II)

(R5O)c(R6)dSi-(A)e—[NR7-(A′)]f-[O-(A″)]g—[NR8-(A′″)]h-Si(R6′)d′(OR5′)c′ (II).

The organosilicon compounds of formula (II) each bear at their two ends the silicon-comprising groupings (R5O)c(R6)dSi— and —Si(R6′)d′(OR5′)c′,

In the central part of the molecule of formula (II) there are the groups -(A)e- and —[NR7-(A′)]f-

and —[O-(A″)]g- and —[NR8-(A′″)]h-. Here, each of the radicals e, f, g, and h can independently of one another stand for the number 0 or 1, with the proviso that at least one of the radicals e, f, g, and h is different from 0. In other words, an organic silicon compound of formula (II) as contemplated herein comprises at least one grouping from the group including comprising -(A)- and —[NR7-(A′)]- and —[O-(A″)]- and —[NR8-(A′″)]-.

In the two terminal structural units (R5O)c(R6)dSii- and —Si(R6′)d′(OR5′)c, the radicals R5, R5′, R5″ independently of one another represent a hydrogen atom or a C1-C6 alkyl group. The radicals R6, R6′ and R6″ independently represent a C1-C6 alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeing's with the best wash fastness values could be obtained if the residues c and c′ both stand for the number 3. In this case d and d′ both stand for the number 0.

In another preferred embodiment, the method is wherein the agent (a) comprises at least one organic silicon compound of formula (II),

(R5O)c(R6)dSi-(A)e-[NR7-(A′)]f-[O-(A″)]g-[NR8-(A′″)]h-Si(R6′)d′(OR5′)c′ (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the invention corresponds to formula (IIa)

(R5O)3Si-(A)e-[NR7-(A′)]f-[O-(A″)]g-[NR8-(A″′)]h-Si(OR5′)3 (IIa).

The radicals e, f, g, and h can independently stand for the number 0 or 1, whereby at least one radical from e, f, g, and h is different from zero. The abbreviations e, f, g, and h thus define which of the groupings -(A)e- and —[NR7-(A′)]f- and —[O-(A″)]g- and —[NR8-(A′″)]h- are in the middle part of the organic silicon compound of formula (II).

In this context, the presence of certain groupings has proved to be particularly beneficial in terms of increasing washability. Particularly good results were obtained when at least two of the residues e, f, g, and h stand for the number 1. Especially preferred e and f both stand for the number 1. Furthermore, g and h both stand for the number 0.

If e and f both stand for the number 1 and g and h both stand for the number 0, the organic silicon compounds correspond to formula (IIb)

(R5O)c(R6)dSi-(A)-[NR7-(A′)]-Si(R6′)d′(OR5′)c′ (IIb).

The radicals A, A′, A″, A′″ and A″″ independently represent a linear or branched divalent C1-C20 alkylene group. Preferably the radicals A, A′, A″, A′″ and A″″ independently of one another represent a linear, divalent C1-C20 alkylene group. Further preferably the radicals A, A′, A″, A′″ and A″″ independently represent a linear divalent C1-C6 alkylene group. In particular, the radicals A, A′, A″, A′″ and A″″ independently of one another represent a methylene group (—CH2-), an ethylene group (—CH2-CH2-), a propylene group (—CH2-CH2-CH2-) or a butylene group (—CH2-CH2-CH2-CH2-). In particular, the radicals A, A′, A″, A′″ and A″″ stand for a propylene group (—CH2-CH2-CH2-).

If the radical f represents the number 1, then the organic silicon compound of formula (II) as contemplated herein comprises a structural grouping —[NR7-(A′)]-.

If the radical f represents the number 1, then the organic silicon compound of formula (II) as contemplated herein comprises a structural grouping —[NR8-(A′″)]-.

Wherein R7 and R7 independently represent a hydrogen atom, a C1-C6 alkyl group, a hydroxy-C1-C6 alkyl group, a C2-C6 alkenyl group, an amino-C1-C6 alkyl group or a group of the formula (III)

-(A″″)-Si(R6″)d″(OR5″)c″ (III).

Very preferably, R7 and R8 independently represent a hydrogen atom, a methyl group, a 2-hydroxyethyl group, a 2-alkenyl group, a 2-aminoethyl group or a grouping of formula (III).

When the radical f represents the number 1 and the radical h represents the number 0, the organic silicon compound as contemplated herein comprises the grouping [NR7-(A′)] but not the grouping —[NR8-(A″)]. If the radical R7 now stands for a grouping of the formula (III), the agent (a) comprises an organic silicon compound with 3 reactive silane groups.

In another preferred embodiment, the method is wherein the agent (a) comprises at least one organic silicon compound of formula (II),

(R5O)c(R6)dSi-(A)e-[NR7-(A′)]f-[O-(A″)]g-[NR8-(A′″)]h-Si(R6′)d′(OR5′)c′ (II),

where
e and f both stand for the number 1,
g and h both stand for the number 0,
A and A′ independently represent a linear, divalent C1-C6 alkylene group, and
R7 represents a hydrogen atom, a methyl group, a 2-hydroxyethyl group, a 2-alkenyl group, a 2-aminoethyl group or a group of formula (III).

In a further preferred embodiment, the method is wherein the agent (a) comprises at least one organic silicon compound of formula (II), wherein
e and f both stand for the number 1,
g and h both stand for the number 0,
A and A′ independently of one another represent a methylene group (—CH2-), an ethylene group (—CH2-CH2-) or a propylene group (—CH2-CH2-CH2), and
R7 represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem are

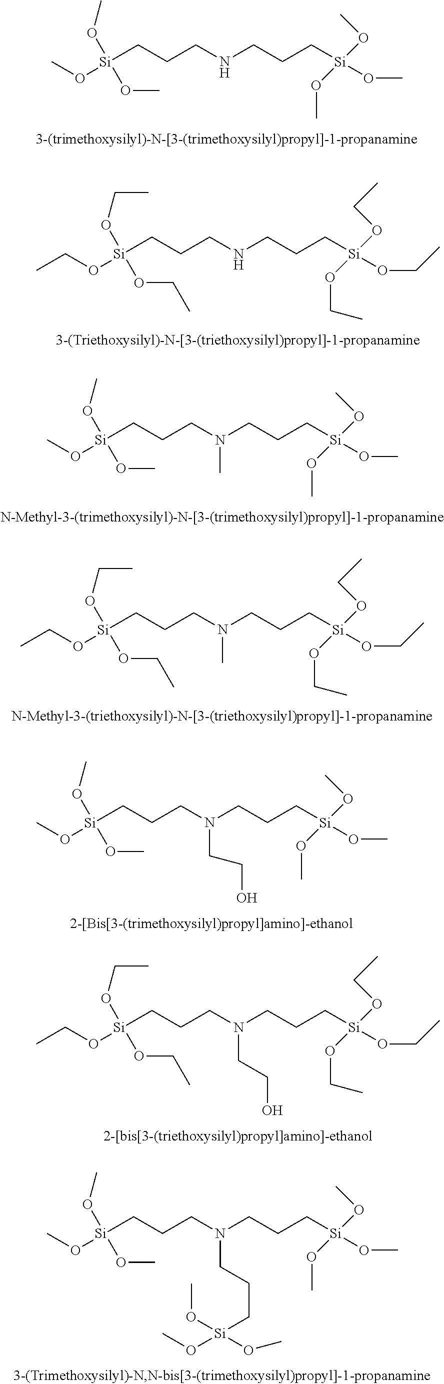

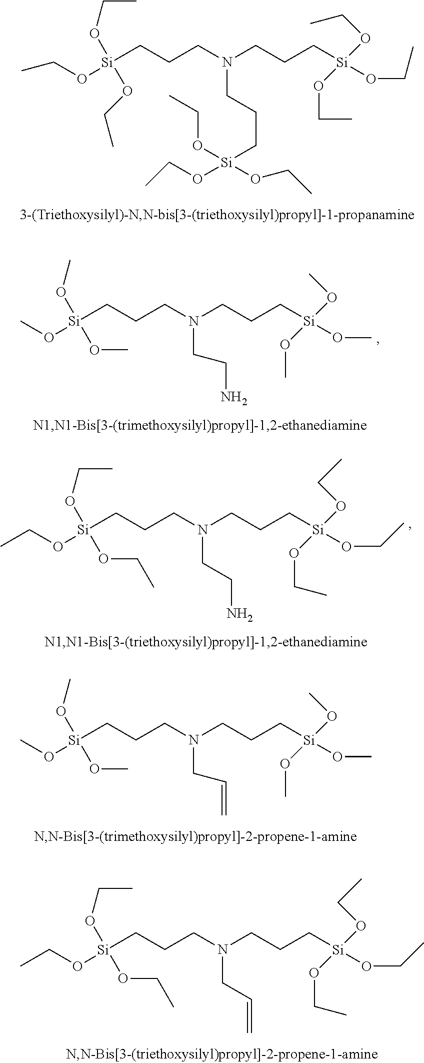

The organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amines with the CAS number 13497-18-2 can be purchased from Sigma-Aldrich, for example.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

In another preferred embodiment, an agent (a) is wherein it comprises at least one organic silicon compound of the formula (II) including comprising selected from the group of
3-(trimethoxysilyl)-N-[3-(trimethoxysilyl) propyl]-1-propanamine
3-(Triethoxysilyl)-N-[3-(triethoxysilyl) propyl]-1-propanamine
N-methyl-3-(trimethoxysilyl)-N-[3-(trimethoxysilyl) propyl]-1-propanamine
N-Methyl-3-(triethoxysilyl)-N-[3-(triethoxysilyl) propyl]-1-propanamine
2-[Bis[3-(trimethoxysilyl) propyl]amino]-ethanol
2-[bis[3-(triethoxysilyl) propyl]amino]ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-bis[3-(trimethoxysilyl) propyl]-1,2-ethanediamine,
N1,N1-bis[3-(triethoxysilyl) propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

In further dyeing trials, it has also been found to be particularly advantageous if the agent (a) applied to the keratinous material in the process comprises at least one organic silicon compound of the formula (IV)

R9Si(OR10)k(R11)m (IV).

The organic silicon compound(s) of formula (IV) may also be referred to as silanes of the alkyl alkoxysilane or alkyl hydroxy silane type,

R9Si(OR10)k(R11)m (IV),

where
R9 stands for a C1-C18 alkyl group,
R10 represents a hydrogen atom or a C1-C6 alkyl group,
R11 represents a C1-C6 alkyl group
k is an integer from 1 to 3, and
m stands for the integer 3-k.

In another preferred embodiment, the method is wherein the agent (a) comprises at least one organic silicon compound of formula (IV).

R9Si(OR10)k(R11)m (IV),

where
R9 stands for a C1-C18 alkyl group,
R10 represents a hydrogen atom or a C1-C6 alkyl group,
R11 represents a C1-C6 alkyl group
k is an integer from 1 to 3, and
m stands for the integer 3-k.

In a further preferred embodiment, the method is wherein the agent (a) comprises, in addition to the organic silicon compound or compounds of formula (I), at least one further organic silicon compound of formula (IV)

R9Si(OR10)k(R11)m (IV),

where
R9 stands for a C1-C18 alkyl group,
R10 represents a hydrogen atom or a C1-C6 alkyl group,
R11 represents a C1-C6 alkyl group
k is an integer from 1 to 3, and
m stands for the integer 3-k.

In a further preferred embodiment, the method is wherein the agent (a) comprises, in addition to the organic silicon compound or compounds of formula (II), at least one further organic silicon compound of formula (IV)

R9Si(OR10)k(R11)m (IV),

where
R9 stands for a C1-C18 alkyl group,
R10 represents a hydrogen atom or a C1-C6 alkyl group,
R11 represents a C1-C6 alkyl group
k is an integer from 1 to 3, and
m stands for the integer 3-k.

In a further preferred embodiment, the process is wherein the agent (a) comprises, in addition to the organic silicon compound or compounds of the formula (I) and/or (II), at least one further organic silicon compound of the formula (IV)

R9Si(OR10)k(R11)m (IV),

where
R9 stands for a C1-C18 alkyl group,
R10 represents a hydrogen atom or a C1-C6 alkyl group,
R11 represents a C1-C6 alkyl group
k is an integer from 1 to 3, and
m stands for the integer 3-k.

In the organic silicon compounds of formula (IV), the radical R9 represents a C1-C18 alkyl group. This C1-C18 alkyl group is saturated and can be linear or branched. Preferably, R9 represents a linear C1-C18 alkyl group. Preferably, R9 represents a methyl group, an ethyl group, an n-propyl group, an n-butyl group, an n-pentyl group, an n-hexyl group, an n-octyl group, an n-dodecyl group or an n-octadecyl group. Particularly preferably, R9 represents a methyl group, an ethyl group, an n-hexyl group or an n-octyl group.

In the organic silicon compounds of formula (IV), the radical R10 represents a hydrogen atom or a C1-C6 alkyl group. Particularly preferably, R10 represents a methyl group or an ethyl group.

In the organic silicon compounds of form (IV), the radical R11 represents a C1-C6 alkyl group. R11 stands for a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Dyeing's with the best wash fastnesses were obtained when an agent (a) comprising at least one organic silicon compound corresponding to formula (IV): in which the radical k is the number 3, was used in the process. In this case the rest m stands for the number 0.

Organic silicon compounds of the formula (IV) which are particularly suitable for solving the problem as contemplated herein are

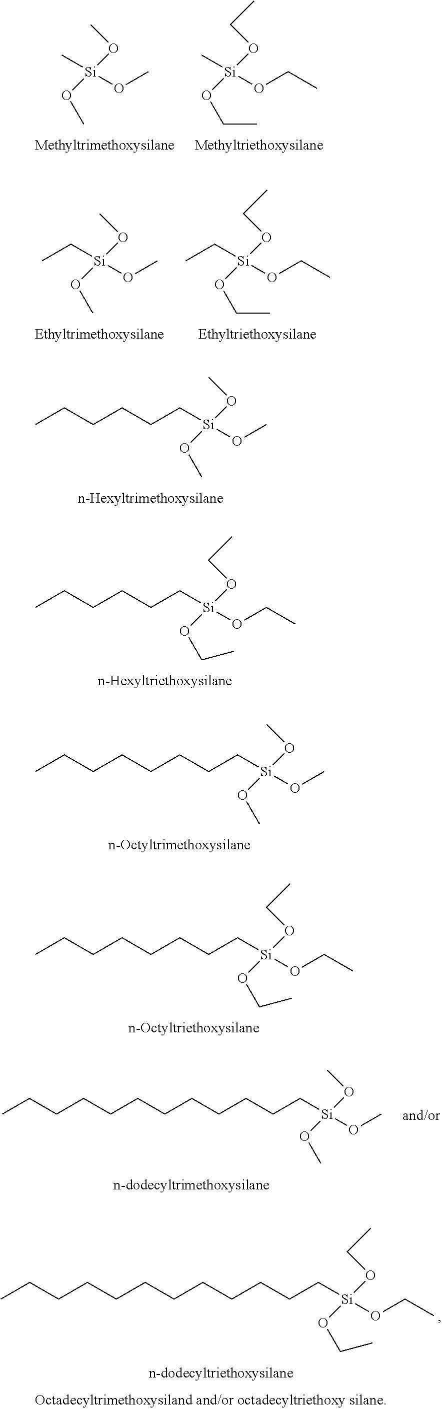

In a further preferred embodiment, the method is wherein the agent (a) comprises at least one organic silicon compound of formula (IV) including comprising selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane
Octadecyltrimethoxysilane and/or
Octadecyltriethoxysilane.

In an explicitly quite particularly preferred embodiment, the process is wherein an agent (a) is applied to the keratinous material, which agent comprises at least one organic silicon compound of the formula (I) including comprising selected from the group of (3-aminopropyl)triethoxysilane and (3-aminopropyl)trimethoxysilane and additionally comprising at least one organic silicon compound of formula (IV) including comprising selected from the group of methyltrimethoxysilane, methyltriethoxysilane, ethyltrimethoxysilane, ethyltriethoxysilane, hexyltrimethoxysilane and hexyltriethoxysilane.

The organic silicon compounds described above are reactive compounds. In this context, it has been found preferable if the agent (a) comprises—based on the total weight of the agent (a)—one or more organic silicon compounds in a total amount of 0.1 to 20% by weight, preferably 0.5 to 15% by weight and particularly preferably 5.0 to 10% by weight.

In this context, it has been found to be particularly preferred if the agent (a) comprises—based on the total weight of the agent (a)—one or more organic silicon compounds of the formula (I) and/or (II) in a total amount of 0.1 to 20% by weight, preferably 0.2 to 15% by weight and particularly preferably 0.2 to 3% by weight.

It has further been found to be particularly preferred if the agent (a) comprises—based on the total weight of the agent (a)—one or more organic silicon compounds of the formula (IV) in a total amount of 0.1 to 20% by weight, preferably 0.5 to 15% by weight and particularly preferably 2 to 8% by weight.

The organosilicon compound may also be present in the agent (a) in the form of condensation products and/or (partial) hydrolysates of the organosilicon compounds. For example, the condensation products may include the condensation products of two, three or four organosilicon compounds.

Particularly resistant strains could be obtained when using an alkaline adjusted agent (a). Preferably, agent (a) comprises water and has a pH of from 7 to 11.5, preferably from 7.5 to 11, and more preferably from 8 to 10.5.

In another very particularly preferred embodiment, the process is wherein the agent (a) has a pH of from 7 to 11.5, preferably from 7.5 to 11, and particularly preferably from 8 to 10.5.

Agent (b)

The agent (b) is characterized by the presence of at least one colorant compound including comprising selected from the group of pigments and/or direct dyes. The agent (b) may also be called colorant (b).

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

Suitable color pigments can be of inorganic and/or organic origin.

In a preferred embodiment, the agent (b) is wherein it comprises at least one colorant compound including comprising selected from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-comprising silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, the process is wherein the agent (b) comprises at least one colorant compound including comprising selected from the group of colored metal oxides, metal hydroxides, metal oxide hydrates, silicates, metal sulfides, complex metal cyanides, metal sulfates, bronze pigments and/or mica- or mica-based colored pigments coated with at least one metal oxide and/or a metal oxychloride.

In a further preferred embodiment, agent (b) is wherein it comprises at least one colorant compound from the group of pigments selected from mica- or mica-based pigments which are reacted with one or more metal oxides including comprising selected from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment, the agent (b) may also comprise one or more colorant compounds including comprising selected from the group of organic pigments.

The organic pigments are correspondingly insoluble organic dyes or colorants which may be selected, for example, from the group of nitroso, nitro-azo, xanthene, anthraquinone, isoindolinone, isoindoline, quinacridone, perinone, perylene, diketopyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, the process is wherein the composition (b) comprises at least one colorant compound from the group of organic pigments including comprising selected from the group of carmine, quinacridone, phthalocyanine, sorghum, blue pigments having the Color Index numbers CI 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof.

The organic pigment can also be a color paint. As contemplated herein, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent light and temperature resistance, the use of the above pigments in the agent (b) of the process is particularly preferred. It is also preferred if the pigments used have a certain particle size. As contemplated herein, it is advantageous if the at least one pigment has a mean particle size D50 of from 1 to 50 μm, preferably from 5 to 45 μm, preferably from 10 to 40 μm, from 14 to 30 μm. The mean particle size D50, for example, can be determined using dynamic light scattering (DLS).

The pigment or pigments may be used in an amount of 0.001 to 20% by weight, of 0.05 to 5% by weight, each based on the total weight of agent (b).

As colorant compounds, the agents (b) used in the process may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments.

Preferably, the direct dyes within the meaning of the present disclosure have a solubility in water (760 mmHg) at 25° C. of more than 1.0 g/L.

Direct dyes can be divided into anionic, cationic, and non-ionic direct dyes.

In a further preferred embodiment, an agent (b) is wherein it comprises at least one anionic, cationic and/or nonionic direct dye as the coloring compound.

In another preferred embodiment, an agent (b) is wherein it comprises at least one anionic, cationic and/or nonionic direct dye.

Suitable cationic direct dyes include Basic Blue 7, Basic Blue 26, Basic Violet 2, and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51, and/or Basic Red 76

As non-ionic direct dyes, non-ionic nitro and quinone dyes and neutral azo dyes can be used. Suitable non-ionic direct dyestuffs are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

In a further preferred embodiment, the process is wherein the agent (b) comprises at least one direct dye including comprising selected from the group of anionic, cationic, and nonionic direct dyes.

In the course of the work leading to the present disclosure, it has been found that dyeing's of particularly high color intensity can be produced with agents (b) comprising at least one anionic direct dye.

In an explicitly quite particularly preferred embodiment, an agent (b) used in the process is therefore wherein it comprises at least one anionic direct dye.

Anionic direct dyes are also called acid dyes. Acid dyes are direct dyes that have at least one carboxylic acid group (—COOH) and/or one sulphonic acid group (—SO3H). Depending on the pH value, the protonated forms (—COOH, —SO3H) of the carboxylic acid or sulphonic acid groups are in equilibrium with their deprotonated forms (—COO—, —SO3- present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Inventive acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L.

The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential characteristic of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In one embodiment of the process, the use of an agent (b) which is wherein it comprises at least one anionic direct dye including comprising selected from the group of nitrophenylenediamines, nitroaminophenols, azo dyes, anthraquinone dyes, triarylmethane dyes, xanthene dyes, rhodamine dyes, oxazine dyes and/or indophenol dyes is thus preferred, the rhodamine dyes, the oxazine dyes and/or the indophenol dyes, the dyes from the abovementioned group each having at least one carboxylic acid group (—COOH), a sodium carboxylate group (—COONa), a potassium carboxylate group (—COOK), a sulfonic acid group (—SO3H), a sodium sulfonate group (—SO3Na) and/or a potassium sulfonate group (—SO3K).

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I.14720), Acid Red 18 (E124, Red 18; CI 16255), Acid Red 27 (E 123, CI 16185, C-Rot 46, Real Red D, FD&C Red Nr.2, Food Red 9, Naphthol red S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent blue V, CI 42051), Acid Blue 7 (CI 42080), Acid Blue 104 (CI 42735), Acid Blue 9 (E 133, Patent blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The water solubility of direct dyes can be determined, for example, in the following way. 0.1 g of the direct dye is added to a beaker. A stir-fish is added. Then add 100 ml of water. This mixture is heated to 25° C. on a magnetic stirrer while stirring. It is stirred for 60 minutes. The aqueous mixture is then visually assessed. If there are still undissolved residues, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).

Acid Yellow 3 is a mixture of the sodium salts of mono- and disulfonic acids of 2-(2-quinolyl)-1H-indene-1,3(2H)-dione and has a water solubility of 20 g/L (25° C.).

Acid Yellow 9 is the disodium salt of 8-hydroxy-5,7-dinitro-2-naphthalenesulfonic acid, its solubility in water is above 40 g/L (25° C.).

Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.

Acid Orange 7 is the sodium salt of 4-[(2-hydroxy-1-naphthyl)azo]benzene sulphonate. Its water solubility is more than 7 g/L (25° C.).

Acid Red 18 is the trisodium salt of 7-hydroxy-8-[(E)-(4-sulfonato-1-naphthyl)-diazenyl)]-1,3-naphthalenedisulfonate and has a very high-water solubility of more than 20% by weight.

Acid Red 33 is the disodium salt of 5-amino-4-hydroxy-3-(phenylazo)-naphthalene-2,7-disulphonate, its solubility in water is 2.5 g/L (25° C.).

Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).

Acid Blue 9 is the disodium salt of 2-({4-[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a solubility in water of more than 20% by weight (25° C.).

A very particularly preferred process is wherein the agent (b) comprises at least one anionic direct dye including comprising selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2, D&C Brown 1 and mixtures it contain.

The direct dye(s), in particular the anionic direct dyes, can be used in different amounts in the agent (b) depending on the desired color intensity. Particularly good results could be obtained if the agent (b)—based on the total weight of the agent (b)—comprises one or more direct dyes in a total amount of 0.01 to 10% by weight, preferably 0.1 to 8% by weight, more preferably 0.2 to 6% by weight and very preferably 0.5 to 4.5% by weight.

In a further preferred embodiment, an agent (b) is wherein it comprises—based on the total weight of the agent (b)—one or more direct dyes in a total amount of from 0.01 to 10% by weight, preferably from 0.1 to 8% by weight, more preferably from 0.2 to 6% by weight and very particularly preferably from 0.5 to 4.5% by weight.

In a further preferred embodiment, the agent (b) is wherein it comprises—based on the total weight of the agent—one or more anionic direct dyes in a total amount of from 0.01 to 10% by weight, preferably from 0.1 to 8% by weight, more preferably from 0.2 to 6% by weight and very particularly preferably from 0.5 to 4.5% by weight.

Agent (c)

The agent (c) may be referred to as an after-treatment agent. Agent (c) is characterized by the presence of two selected silicones.

The silicone oils included in agent (c) comprise Si—O repeating units, where the Si atoms may carry organic radicals such as alkyl groups or substituted alkyl groups.

The silicones included in agent (c) are polymeric compounds whose molecular weight is at least 500 g/mol, preferably at least 1000 g/mol, further preferably at least 2500 g/mol, and particularly preferably of at least 5000 g/mol. The silicone oils included in agent (c) are therefore different from the organic silicon compounds of agent (a).

The two silicones are hydroxy terminated, which means that each of the two silicones has at least one terminal OH group.

In other words, an object of the application is a method for coloring keratinous material, in particular human hair, comprising the following steps:
Application of an agent (a) to the keratinous material, wherein the agent (a) comprises at least one organic silicon compound having one, two or three silicon atoms,
Application of an agent (b) to the keratinous material, the agent (b) comprising at least one colorant compound including comprising selected from the group of pigments and/or direct dyes,
Application of an agent (c) to the keratinous material, the agent (c) comprising at least two polymeric silicones.

Another object of the application is, in other words, a method for coloring keratinous material, in particular human hair, comprising the following steps:
Application of an agent (a) to the keratinous material, wherein the agent (a) comprises at least one organic silicon compound having one, two or three silicon atoms,
Application of an agent (b) to the keratinous material, the agent (b) comprising at least one colorant compound including comprising selected from the group of pigments and/or direct dyes,
Application of an agent (c) to the keratinous material, wherein the agent (c) comprises:
(c1) a hydroxy terminated polyorganosiloxane, and
(c2) a reaction product of a hydroxy terminated polyorganosiloxane with an acid and/or an alcohol and/or a wax,

In the course of the work leading to the present disclosure, it was found that the silicones used in agent (c) can have a strong influence on the washout resistance of the colored keratinous material (or hair).

In the process, the application of agent (a) first creates a film of organosilicon compounds (i.e., silanes) on the keratinous material, which has an extremely high affinity for the keratinous material. In interaction with the colorant compounds of agent (b), a layer of colorant compounds is now formed on the keratinous material, the colorant compounds being fixed to the keratinous material by the layer formed by the organic silicon compound(s) of agent (a). It has been found that the layers or films formed on the keratinous material are stabilized by the hydroxy terminated polyorganosiloxanes included in agent (c). Without wishing to be bound by this theory, it is believed that the hydroxy terminated polyorganosiloxanes form covalent bonds with the organic silicon compound(s) or their reaction products included in the formed layer.

Suitable hydroxy terminated polyorganosiloxanes are those with the INCI designation dimethiconol.

It has been found to be particularly advantageous if the agent (c) is, as hydroxy terminated polyorganosiloxane (c1), a polyorganosiloxane of the formula (I)

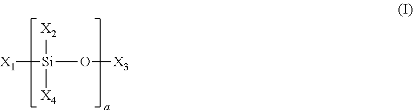

### Includes,

wherein
X1 and X2 are independently OH, OR1, R2, O-PDMS or O-fSiloxane,
X3 is hydrogen or a monovalent hydrocarbon radical having 1 to 8 carbon atoms per radical,
PDMS or fSiloxane,
X4 is a remainder of the formula

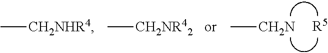

and
a is a number from 1 to 100,
where
R1 is an alkyl radical having 1 to 8 carbon atoms,
R2 is a monovalent, saturated, or unsaturated hydrocarbon radical which is optionally substituted by the elements N, P, S, O, Si, and halogen and has 1 to 200 carbon atoms per radical,
PDMS stands

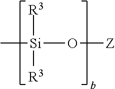

for,
fSiloxan stands

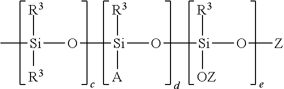

for,
R3 independent of one another is in each case a monovalent saturated or unsaturated hydrocarbon radical having 1 to 200 carbon atoms per radical and optionally substituted by the elements N, P, S, O, Si, and halogen,
A is a radical of the formula R6-[NR7-R8-]fNR72,
where
R6 is a divalent linear or branched hydrocarbon radical comprising 3 to 18 carbon atoms,
R7 is a hydrogen atom, an alkyl radical having 1 to 8 carbon atoms or an acyl radical,
R8 is a divalent hydrocarbon radical comprising 1 to 6 carbon atoms,
b is a number from 1 to 2000,
c is 0 or a number from 1 to 2000,
d is a number from 1 to 1000,
e is 0 or a number from 1 to 5,
f is 0, 1, 2, 3 or 4,
Z is hydrogen, an alkyl radical having from 1 to 8 carbon atoms

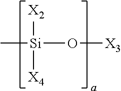

or,
R4 is a monovalent hydrocarbon radical optionally comprising N and/or O atoms and having 1 to 18 carbon atoms, and
R5 is a divalent hydrocarbon radical optionally comprising N and/or O atoms and having 3 to 12 carbon atoms,
with the proviso that the polyorganosiloxane of formula (I) has at least one terminal OH group.

In the context of a further explicitly quite particularly preferred embodiment, a process as contemplated herein is wherein the agent (c) comprises at least one polyorganosiloxane of the formula (I)

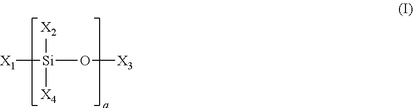

### Includes,

wherein
X1 and X2 are independently OH, OR1, R2, O-PDMS or O-fSiloxane,
X3 is hydrogen or a monovalent hydrocarbon radical having 1 to 8 carbon atoms per radical,
PDMS or fSiloxane,
X4 is a remainder of the formula

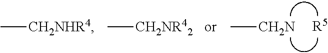

and
a is a number from 1 to 100,
where
R1 is an alkyl radical having 1 to 8 carbon atoms,
R2 is a monovalent, saturated, or unsaturated hydrocarbon radical which is optionally substituted by the elements N, P, S, O, Si, and halogen and has 1 to 200 carbon atoms per radical,
PDMS stands

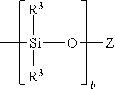

for,
fSiloxan stands

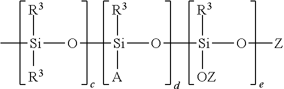

for,
R3 independent of one another is in each case a monovalent saturated or unsaturated hydrocarbon radical having 1 to 200 carbon atoms per radical and optionally substituted by the elements N, P, S, O, Si, and halogen,
A is a radical of the formula R6-[NR7-R8-]fNR72,
where
R6 is a divalent linear or branched hydrocarbon radical comprising 3 to 18 carbon atoms,
R7 is a hydrogen atom, an alkyl radical having 1 to 8 carbon atoms or an acyl radical,
R8 is a divalent hydrocarbon radical comprising 1 to 6 carbon atoms,
b is a number from 1 to 2000,
c is 0 or a number from 1 to 2000,
d is a number from 1 to 1000,
e is 0 or a number from 1 to 5,
f is 0, 1, 2, 3 or 4,
Z is hydrogen, an alkyl radical having from 1 to 8 carbon atoms

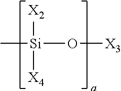

or,
R4 is a monovalent hydrocarbon radical optionally comprising N and/or O atoms and having 1 to 18 carbon atoms, and
R5 is a divalent hydrocarbon radical optionally comprising N and/or O atoms and having 3 to 12 carbon atoms,
with the proviso that the polyorganosiloxane of the formula (I) has at least one terminal OH group, as hydroxy terminated polyorganosiloxane.

Examples of an alkyl radical R1 are methyl, ethyl, n-propyl, iso-propyl, 1-n-butyl, 2-n-butyl, iso-butyl, tert-butyl, n-pentyl, iso-pentyl, neo-pentyl, tert-. pentyl, n-hexyl, n-heptyl, n-octyl, iso-octyl or 2,2,4-trimethylpentyl-, with methyl-, ethyl- and butyl- being preferred.

Examples of hydrocarbon radicals R2 and R3 include alkyl radicals such as methyl, ethyl, n-propyl, iso-propyl, 1-n-butyl, 2-n-butyl, iso-butyl, tert-butyl, n-pentyl, iso-pentyl, neo-pentyl, tert-. Pentyl, n-hexyl, n-heptyl, n-octyl, iso-octyl, 2,2,4-trimethylpentyl, n-nonyl, n-decyl, n-dodecyl, n-octadecyl, cyclopentyl, cyclohexyl, cycloheptyl, methylcyclohexyl, vinyl, 5-hexenyl, cyclohexenyl, 1-propenyl, allyl, 3-butenyl, 4-pentenyl, phenyl, naphthyl, anthryl, phenanthryl, o-tolyl, m-tolyl, p-tolyl, xylyl, ethylphenyl, benzyl, alpha-phenylethyl and beta-phenylethyl. Preferred radicals R2 are the methyl, ethyl, octyl and phenyl radicals, and particularly preferred are the methyl and ethyl radicals.

Examples of halogenated radicals R2 and R3 include the 3,3,3-trifluoro-n-propyl, 2,2,2,2′,2′,2′-hexafluoroisopropyl, heptafluoroisopropyl, o-chlorophenyl, m-chlorophenyl and p-chlorophenyl radicals.

Examples of R4 include the alkyl, cycloalkyl, aryl, alkaryl and aralkyl radicals listed for hydrocarbon radicals R2 and R3.

Preferred examples of R5 are radicals of the formulae —CH2-CH2-O—CH2-CH2-, —CH2-CH2-NH—CH2-CH2- or —CH2-CH2-NH—CH2-, the radical —CH2-CH2-O—CH2-CH2-being particularly preferred.

Examples of R6 are alkylene radical with 3 to 10 carbon atoms such as propylene, butylene, pentylene, hexylene, heptylene, octylene, nonylene and decylene.

R7 may be hydrogen, methyl, ethyl, n-propyl, iso-propyl, 1-n-butyl, 2-n-butyl, iso-butyl, tert-butyl, n-pentyl, iso-pentyl, neo-pentyl, tert-. pentyl, n-hexyl, n-heptyl, n-octyl, iso-octyl, 2,2,4-trimethylpentyl or acetyl, with a hydrogen atom being preferred.

Preferred examples of R8 include alkylene radicals such as methylene, ethylene, propylene, butylene, pentylene, or hexylene.

Z is preferably hydrogen or methyl, ethyl, n-propyl, iso-propyl, 1-n-butyl, 2-n-butyl, iso-butyl, tert-butyl, n-pentyl, iso-pentyl, neo-pentyl, tert-. pentyl, n-hexyl, n-heptyl, n-octyl, iso-octyl or 2,2,4-trimethylpentyl-, with hydrogen, methyl-, ethyl- and butyl- being particularly preferred.

Preferred radicals X4 are, according to the above definitions for R4 and R5, aminomethyl-, methylaminomethyl-, dimethylaminomethyl-, diethylaminomethyl-, dibutylaminomethyl-, cyclohexylaminomethyl-, morpholinomethyl-, piperidinomethyl-, piperazinomethyl-, ((diethoxymethyl silyl)methyl)cyclohexylaminomethyl-, ((triethoxysilyl)methyl)cyclohexylaminomethyl-, anilinomethyl-, 3-dimethylaminopropyl-aminomethyl-, bis(3-dimethylaminopropyl)aminomethyl- and mixtures thereof. In this context, it is highly preferred if the cosmetic agent comprises polyorganosiloxanes of the formula (I) which contain the morpholinomethyl radical as radical X4.

According to the definitions for R6, R7 and R8 are preferred examples of residue A:
—(CH2)3NH2
—(CH2)3-NH—(CH2)2-NH2
—CH2CH(CH3)CH2-NH—(CH2)2-NH2
—(CH2)3-NH(Cyclohexyl)
—(CH2)3-NHCH3
—(CH2)3-N(CH3)2
—(CH2)3-NHCH2CH3
—(CH2)3-N(CH2CH3)2
—(CH2)4-NH2
—CH2CH(CH3)CH2-NH2
—(CH2)3-NH—(CH2)2-NHCH3
—(CH2)3-NH—(CH2)2-N(CH3)2
—(CH2)3-NH—(CH2)2-NHCH2CH3
—(CH2)3-NH—(CH2)2-N(CH2CH3)2
—(CH2)3[-NH—CH2CH2]2-NH2
—(CH2)3-NH(Acetyl)
—(CH2)3-NH—(CH2)2-NH(Acetyl) and
—(CH2)3-N(Acetyl)-(CH2)2-NH(Acetyl).

For the preparation of the polyorganosiloxanes of the formula (I), preferably commercially available polydimethylsiloxanes with terminal silanol groups and/or polydimethylsiloxanes with terminal alkoxy and silanol groups and/or amine-functionalized siloxanes comprising silanol groups or alkoxy and silanol groups are reacted with a dialkoxy and/or trialkoxysilane comprising a radical of the formula

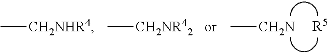

has been implemented.

Accordingly, in formula (I), “fSiloxane” represents a radical derived from an amine-functionalized siloxane.

Trialkoxysilanes or a mixture of dialkoxy- and trialkoxysilanes, are particularly preferred, with the use of trialkoxysilanes alone being especially preferred. When trialkoxysilanes or a mixture of dialkoxy- and trialkoxysilanes are used, at least partially crosslinked polyorganosiloxanes are obtained, regardless of the structure of the siloxanes used and the position of the alkoxy and/or silanol groups in the siloxanes. In a very particularly preferred embodiment, the cosmetic agent comprises crosslinked polyorganosiloxanes. In a highly preferred embodiment, the cosmetic composition comprises crosslinked polyorganosiloxanes derived from the reaction of siloxanes and trialkoxysilanes.

Preferred examples of the dialkoxy or trialkoxysilanes used include:

### Diethylaminomethylmethyldimethoxysilane,

### Dibutylaminomethyltriethoxysilane,

### Dibutylaminomethyltributoxysilane,

### Cyclohexylaminomethyltrimethoxysilane,

### Cyclohexylaminomethyltriethoxysilane,

### Cyclohexylaminomethyl-methyldiethoxysilane,

### Anilinomethyltriethoxysilane,

### Anilinomethylmethyldiethoxysilane,

### Morpholinomethyltriethoxysilane,

### Morpholinomethyltrimethoxysilane,

### Morpholinomethyltriisopropoxysilane,

### 3-Dimethylaminopropyl-aminomethyltrimethoxysilane,

### Morpholinomethyltributoxysilane,

Morpholinomethyltrialkoxysilane, wherein the alkoxy radical is a C1-C4 alkoxy radical, a mixture of methoxy and ethoxy radicals,

### Piperazinomethyltriethoxysilane,

Piperidinomethyltriethoxysilane and Partial hydrolysates thereof.

A particularly preferred silane is morpholinomethyltriethoxysilane.

A particularly preferred amine-functionalized siloxane is a copolymer of 3-(2-aminoethylamino)propylmethylsiloxy and dimethylsiloxy units, which has silanol groups or alkoxy and silanol groups.

A cosmetic composition is particularly preferred in which at least one compound known under the INCI name amodimethicone/morpholinomethyl silsesquioxane copolymer is used as the hydroxy terminated polyorganosiloxane. This polyorganosiloxane is commercially available under the name Belsil® ADM 8301 E (ex Wacker). The raw material is a microemulsion and has the following components: Amodimethicone/Morpholinomethyl Silsesquioxane Copolymer, Trideceth-5, Glycerin, Phenoxyethanol and Water.

In the context of a further explicitly quite particularly preferred embodiment, the method is wherein the agent (c) comprises a compound known under the INCI designation amodimethicone/morpholinomethyl silsesquioxane copolymer as hydroxy terminated polyorganosiloxane (c1).

Composition (c) comprises the hydroxy terminated polyorganosiloxane (c1) in an amount of from 0.1 to 10% by weight, preferably from 0.1 to 8% by weight, more preferably from 0.125 to 6% by weight, still more preferably from 0.15 to 4% by weight and very particularly preferably from 0.2 to 2% by weight, in each case based on the weight of cosmetic composition (c).

As a second ingredient (c2), the agent (c) comprises the reaction product of a hydroxy terminated polyorganosiloxane with an acid and/or an alcohol and/or a wax.

The acid, alcohol or wax react with the terminal hydroxyl group(s) polyorganosiloxane to form, for example, ester or ether.

Within the scope of a further explicitly quite particularly preferred embodiment, the method is wherein the agent (c) comprises, as reaction product of a hydroxy-terminated polyorganosiloxane with an acid and/or an alcohol and/or a wax (c2), a reaction product of a hydroxy-terminated polyorganosiloxane with an acid, which is including comprising selected from the group of reaction products of a hydroxy-terminated polyorganosiloxane with a fatty acid, reaction products of a hydroxy-terminated polyorganosiloxane with an amino acid, reaction products of a hydroxy-terminated polyorganosiloxane with an α-hydroxy acid, and mixtures thereof.

Preferably, agent (c) comprises as ingredient (c2) the reaction product of a hydroxy terminated polyorganosiloxane with a fatty acid.

Accordingly, in the context of a further explicitly quite particularly preferred embodiment, the process is wherein the agent (c) comprises, as reaction product of a hydroxy-terminated polyorganosiloxane with an acid and/or an alcohol and/or a wax (c2), a reaction product of a hydroxy-terminated polyorganosiloxane with a fatty acid.

In the context of the present disclosure, fatty acids are aliphatic carboxylic acids comprising unbranched or branched, optionally hydroxylated, hydrocarbon radicals having 4 to 40, preferably 8 to 24, carbon atoms. The fatty acids used in the present disclosure can be both naturally occurring and synthetically produced fatty acids. Furthermore, the fatty acids can be mono- or polyunsaturated. The fatty acid may also comprise a mixture of several fatty acids.

Particularly preferred fatty acids are including comprising selected from the group of behenic acid, fatty acids derived from the seed oil of borage (Borago officinalis L.), fatty acids derived from Vateria indica, 12-hydroxystearic acid, isostearic acid, fatty acids derived from meadowfoam seed oil, fatty acids of mohwa butter, fatty acids derived from salbutter, fatty acids derived from coconut butter, fatty acids derived from illipe butter, stearic acid, and mixtures thereof.

Reaction products of a hydroxy-terminated polyorganosiloxane with a fatty acid include, for example, the reaction product of dimethiconol with behenic acid (INCI: dimethiconol behenate), the reaction product of dimethiconol with fatty acids obtained from the seed oil of borage (Borago officinalis L.) (INCI: dimethiconol borageate), the reaction product of dimethiconol with fatty acids obtained from Vateria Indica (INCI: dimethiconol dhupa butterate), the reaction product of dimethiconol with 12-hydroxystearic acid (INCI: dimethiconol hydroxystearate), the reaction product of dimethiconol with isostearic acid (INCI: dimethiconol isostearate), the reaction product of dimethiconol with fatty acids obtained from the seed oil of American meadowfoam (“meadowfoam seed oil”) (INCI: dimethiconol meadowfoamate), the reaction product of dimethiconol with fatty acids derived from mohwa butter (INCI: dimethiconol mohwa butterate), the reaction product of dimethiconol with fatty acids derived from sal butter (INCI: dimethiconol sal butterate), the reaction product of dimethiconol with fatty acids derived from coconut butter (INCI: dimethiconol kokum butterate), the reaction product of dimethiconol with fatty acids obtained from illipe butter (INCI: dimethiconol illipe butterate) and/or the reaction product of dimethiconol with stearic acid (INCI: dimethiconol stearate).

Most preferably, the fatty acid is including comprising selected from the group of fatty acids derived from meadowfoam seed oil, behenic acid, stearic acid, and mixtures thereof. It is particularly preferred that the fatty acid comprises a mixture of fatty acids derived from American meadowfoam seed oil (“meadowfoam seed oil”). It is highly preferred that the fatty acid is a mixture of fatty acids derived from American meadowfoam seed oil (“meadowfoam seed oil”).

For example, a suitable reaction product of a hydroxy-terminated polyorganosiloxane with a fatty acid is known as Fancorsil LEVI-2 (INCI: Dimethiconol Meadowfoamate) available from Elementis.

In the context of a further explicitly quite particularly preferred embodiment, the method is wherein the agent (c) comprises:

(c1) at least one compound known under the INCI name amodimethicone/morpholinomethyl silsesquioxane copolymer.
(c2) the reaction product of dimethiconol with fatty acids obtained from the seed oil of American meadowfoam seed oil (INCI: dimethiconol meadowfoamate).

Likewise, quite particularly preferred embodiment of the method is wherein the agent (c) comprises:

(c1) at least one compound known under the INCI name amodimethicone/morpholinomethyl silsesquioxane copolymer.
(c2) the reaction product of dimethiconol with stearic acid (INCI: dimethiconol stearate).

Another likewise explicitly quite particularly preferred embodiment of the method is wherein the agent (c) comprises:

(c1) at least one compound known under the INCI name amodimethicone/morpholinomethyl silsesquioxane copolymer.
(c2) the reaction product of dimethiconol with behenic acid (INCI: dimethiconol behenate).

In a further preferred embodiment, the method is wherein the agent (c)—based on the total weight of the agent (c)—is the reaction product of a hydroxy-terminated polyorganosiloxane with an acid and/or an alcohol and/or a wax (c2) in an amount of 0.1 to 10% by weight, preferably 0.2 to 8% by weight, more preferably 0.3 to 6% by weight, even more preferably 0.4 to 4% by weight. -% and particularly preferably from 0.5 to 2% by weight.

Other ingredients in agents (a), (b) and (c)

The previously described agents (a), (b) and (c) may further include one or more optional ingredients.

The products may also contain one or more surfactants. The term surfactants refer to surface-active substances. A distinction is made between anionic surfactants including comprising a hydrophobic residue and a negatively charged hydrophilic head group, amphoteric surfactants, which carry both a negative and a compensating positive charge, cationic surfactants, which in addition to a hydrophobic residue have a positively charged hydrophilic group, and non-ionic surfactants, which have no charges but strong dipole moments and are strongly hydrated in aqueous solution.

Zwitterionic surfactants are those surface-active compounds which carry at least one quaternary ammonium group and at least one —COO(−)- or —SO3(−) group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example, cocoacylaminopropyl dimethyl ammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name cocamidopropyl betaine.

Ampholytic surfactants are surface-active compounds which, in addition to a C8-C24 alkyl or acyl group in the molecule, contain at least one free amino group and at least one —COOH or —SO3H group and can form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to 24 C atoms in the alkyl group. Typical examples of amphoteric or zwitterionic surfactants are alkylbetaines, alkylamidobetaines, amino propionates, aminoglycinate, imidazoliniumbetaines and sulfobetaines.

Particularly preferred ampholytic surfactants are N-cocosalkylaminopropionate, cocosacylaminoethylaminopropionate and C12-C18-acylsarcosine.

The products may also additionally contain at least one non-ionic surfactant. Suitable non-ionic surfactants are alkyl polyglycosides as well as alkylene oxide addition products to fatty alcohols and fatty acids with 2 to 30 mol ethylene oxide per mol fatty alcohol or fatty acid. Preparations with good properties are also obtained if they contain as non-ionic surfactants fatty acid esters of ethoxylated glycerol reacted with at least 2 mol ethylene oxide.

It may be particularly preferred that agent (c) further comprises an alkoxylated fatty alcohol.

For example, the fatty alcohol may be selected from C9-C11 fatty alcohols, C12-C13 fatty alcohols, C12-C15 fatty alcohols, C12-C16 fatty alcohols, C14-C15 fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol and tridecyl alcohol.

In particular, the alkoxy group may comprise ethoxy and/or propoxy and/or butoxy groups. Particularly preferably, the alkoxylated fatty alcohol is an ethoxylated fatty alcohol.

The at least one alkoxy group of the “alkoxylated fatty alcohol” may, for example, be derived from an alkoxylation reaction with alkylene oxide, in particular ethylene oxide and/or propylene oxide.

It is preferred that the alkoxylated fatty alcohol is including comprising selected from the group of Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100, and mixtures thereof.

The designation ceteareth-2, for example, stands for a C16-C18 fatty alcohol with an average of 2 ethylene oxide units per molecule.

In a very particularly preferred embodiment of agent (c), the alkoxylated fatty alcohol comprises ceteareth-20.

The amount of the alkoxylated fatty alcohol is preferably from 0.1 to 5% by weight and more preferably from 0.25 to 3% by weight, in each case based on the total amount of agent (c).

In addition, the products may also contain at least one cationic surfactant. Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually including comprising a hydrocarbon backbone (e.g., including comprising one or two linear or branched alkyl chains) and the positive charge(s) being in the hydrophilic head group. Examples of cationic surfactants are
quaternary ammonium compounds which may carry one or two alkyl chains with a chain length of 8 to 28 carbon atoms as hydrophobic radicals,
quaternary phosphonium salts substituted by one or more alkyl chains having a chain length of 8 to 28 carbon atoms or
tertiary sulfonium salts.

Furthermore, the cationic charge can also be part of a heterocyclic ring (e.g., an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of 0.1 to 45 wt. %, preferably 1 to 30 wt. % and most preferably 1 to 15 wt. %—based on the total weight of the respective agent.

It may be particularly preferred that the agent (c) is free from cationic surfactants since these can have a destabilizing effect. “Free of” means that the agent (c) comprises a maximum of 0.2% by weight and preferably 0% by weight, in each case based on the total weight of the agent (c), of cationic surfactants.

Furthermore, the agents may also contain at least one anionic surfactant. Anionic surfactants are surface-active agents with exclusively anionic charges (neutralized by a corresponding counter cation). Examples of anionic surfactants are fatty acids, alkyl sulphates, alkyl ether sulphates and ether carboxylic acids with 12 to 20 C atoms in the alkyl group and up to 16 glycol ether groups in the molecule.

The anionic surfactants are used in a total quantity of 0.1 to 45 wt. %, preferably 1 to 30 wt. % and most preferably 1 to 15 wt. %—based on the total weight of the respective agent.

To adjust the desired pH, agents (a), (b) and (c) may also contain at least one alkalizing agent and/or acidifying agent. The pH values for the purposes of the present disclosure are pH values measured at a temperature of 22° C.

As alkalizing agents, agents (a), (b) and (c) may contain, for example, ammonia, alkanolamines and/or basic amino acids.

The alkanolamines that can be used in the compositions are preferably selected from primary amines having a C2-C6 alkyl parent carrying at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

Particularly preferred alkanolamines are selected from 2-aminoethan-1-ol and/or 2-amino-2-methylpropan-1-ol. A particularly preferred embodiment is therefore wherein an agent (a), (b) and/or (c) comprises as alkalizing agent an alkanolamine selected from 2-aminoethan-1-ol and/or 2-amino-2-methylpropan-1-ol.

For the purposes of the invention, an amino acid is an organic compound comprising in its structure at least one protonatable amino group and at least one —COOH or one —SO3H group. Preferred amino acids are amino carboxylic acids, especially α-(alpha)-amino carboxylic acids and w-amino carboxylic acids, whereby α-amino carboxylic acids are particularly preferred.

As contemplated herein, basic amino acids are those amino acids which have an isoelectric point pI of greater than 7.

Basic α-amino carboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine, and histidine, especially preferably arginine and lysine. In another particularly preferred embodiment, an agent as contemplated herein is therefore wherein the alkalizing agent is a basic amino acid from the group arginine, lysine, ornithine and/or histidine.

In addition, the agents (a), (b) and/or (c) may contain further alkalizing agents, in particular inorganic alkalizing agents. Applicable inorganic alkalizing agents are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Very particularly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Acidifiers familiar to the skilled person are, for example, organic acids, such as citric acid, acetic acid, maleic acid, lactic acid, malic acid or tartaric acid, and dilute mineral acids, such as hydrochloric acid, sulfuric acid, or phosphoric acid.

It is preferred that the agent (c) has a pH in the range of 2.5 to 6.5, preferably 2.5 to 5.5, particularly preferably 2.5 to 4.5 and especially preferably 2.5 to 3.5. Lactic acid is particularly preferred for adjusting the pH value. The agent (c) comprises the organic acid, in particular lactic acid, preferably in an amount of from 0.1 to 5% by weight and particularly preferably from 0.25 to 3% by weight, in each case based on the weight of compound (c).

Without wishing to be bound by this theory, it is believed that the use of an agent (c) with an acidic to slightly acidic pH value results in a change in the ionic charge in the film formed on the keratinic materials. This change leads to stabilization and hydrophilization of the films formed.

In the context of a further particularly preferred embodiment, the process is wherein the agent (c) has a pH value of 2.5 to 3.5.

The agents (a), (b) and/or (c) may also contain other active ingredients, auxiliaries and additives, such as solvents; fatty ingredients such as C8-C30 fatty acid triglycerides, C8-C30 fatty acid monoglycerides, C8-C30 fatty acid diglycerides and/or hydrocarbons; polymers; structurants such as glucose or sodium chloride, hair conditioning compounds such as phospholipids, for example lecithin and kephalins; perfume oils, dimethyl isosorbide and cyclodextrins; fiber structure-improving active ingredients, in particular mono-di- and oligosaccharides such as glucose, galactose, fructose, fructose and lactose; dyes for coloring the product; anti-dandruff active ingredients such as piroctone olamine, zinc omadine and climbazole; amino acids and oligopeptides; protein hydrolysates on an animal and/or vegetable basis, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; Polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; Fats and waxes such as fatty alcohols, beeswax, montan wax and kerosene's; swelling and penetrating agents such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate as well as PEG-3-distearate; and blowing agents such as propane-butane mixtures, N20, dimethyl ether, CO2 and air.

Very preferably, the agent (b) additionally comprises at least one film-forming polymer. The at least one film-forming polymer is preferably selected from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

It has been shown that the fastness properties, in particular the wash fastness, of the dyed keratinous material can be significantly improved by using a film-forming polymer in the agent (b).

Accordingly, a process is particularly preferred in which an agent (b) is applied to the keratinous material, the agent (b) comprising at least one colorant compound including comprising selected from the group of pigments and/or direct dyes and a film-forming polymer.

Very preferably, the agent (c) additionally comprises at least one fatty alcohol.

For example, the fatty alcohol may be selected from C9-C11 fatty alcohols, C12-C13 fatty alcohols, C12-C15 fatty alcohols, C12-C16 fatty alcohols, C14-C15 fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol and/or tridecyl alcohol.

In a particularly preferred embodiment of agent (c), the fatty alcohol comprises cetearyl alcohol.

The amount of the fatty alcohol is preferably 0.5 to 10% by weight, more preferably 1 to 9% by weight and particularly preferably 2 to 8% by weight, in each case based on the total amount of agent (c).

The selection of these other substances will be made by the specialist according to the desired properties of the agents. Regarding other optional components and the quantities of these components used, explicit reference is made to the relevant manuals known to the specialist. The additional active ingredients and auxiliaries are preferably used in the agents (a), (b) and/or (c) in amounts of from 0.0001 to 25% by weight in each case, from 0.0005 to 15% by weight, based on the total weight of the respective agent.

Process for Dyeing Keratinous Materials

In the process as contemplated herein, agents (a), (b) and (c) are applied to the keratinous materials, to human hair. Thus, the agent (a), (b), and (c) are the ready-to-use means. The agent (a), (b) and (c) are different from each other.

The agent (a), (b) and (c) can in principle be applied simultaneously or successively, with successive application being preferred.

The best results were obtained when agent (a) was applied to the keratinous materials as a pretreatment agent, then agent (b) was applied as a coloring agent, and subsequently agent (c) was applied as a posttreatment agent.

Therefore, a method for dyeing keratinous material, in particular human hair, comprising the following steps in the order given is particularly preferred:
in a first step, applying an agent (a) to the keratinous material, the agent (a) comprising at least one organic silicon compound,
in a second step, applying an agent (b) to the keratinous material, the agent (b) comprising at least one colorant compound including comprising selected from the group of pigments and/or direct dyes,
in a third step, applying an agent (c) to the keratinous material, the agent comprising (c):
   (c1) a hydroxy terminated polyorganosiloxane, and
   (c2) a reaction product of a hydroxy terminated polyorganosiloxane with an acid and/or an alcohol and/or a wax.

Moreover, to impart a high leaching resistance to the dyed keratinous material over a longer period, agents (a), (b) and (c) are particularly preferably applied within one and the same dyeing process, which means that there is a period of a maximum of several hours between the application of agents (a) and (c).

In a further preferred embodiment, the method is wherein agent (a) is applied first, agent (b) is then applied, and agent (c) is then applied, the time between the application of agents (a) and (c) being a maximum of 24 hours, preferably a maximum of 12 hours, and particularly preferably a maximum of 6 hours.

In the process as contemplated herein, the keratinous materials, in particular human hair, are first treated with agent (a). Subsequently, the actual colorant (b)—which comprises the colorant compounds—is applied to the keratinous materials.

Preferably, agent (a) itself does not contain colorants or coloring compounds. A characteristic feature of the pretreatment agent (a) is its content of at least one reactive organic silicon compound. The reactive organic silicon compound(s) (a) functionalize the hair surface as soon as they meet it. In this way a first, still uncolored film is formed. In the second step of the process, a colorant (b) is now applied to the hair. During application of the colorant (b), the colorant compounds interact with the film formed by the organosilicon compounds and are thus bound to the keratinous materials. By applying the aftertreatment agent (c), the properties of the resulting dyeing can be significantly improved, particularly about fastness properties and especially wash fastness.

In the context of a further form of execution, a procedure comprising the following steps in the order indicated is particularly preferred

(1) Application of the agent (a) on the keratinous material,
(2) Allow the agent (a) to act for a period of 10 seconds to 10 minutes, preferably from 10 seconds to 5 minutes,
(3) if necessary, rinse the keratinous material with water,
(4) Application of agent (b) on the keratinous material,
(5) Allow the agent (b) to act for a period of 30 seconds to 30 minutes, preferably from 30 seconds to 10 minutes, and
(6) Rinse the keratinous material with water,
(7) Application of the agent (c) on the keratinous material,
(8) allowing the agent (c) to act for a period of from 30 seconds to 10 minutes, preferably from 30 seconds to 50 minutes; and
(9) Rinse the keratinous material with water.

By rinsing the keratinous material with water in steps (3), (6) and (9) of the process, it is understood as contemplated herein that only water is used for the rinsing process, without the use of other agents different from agents (a), (b) and (c).

In step (1), agent (a) is first applied to the keratinous materials, in particular human hair.

After application, the agent (a) is left to act on the keratinous materials. In this context, exposure times of 10 seconds to 10 minutes, preferably 20 seconds to 5 minutes and most preferably 30 seconds to 2 minutes to the keratinous materials, to human hair, have proven to be particularly advantageous.

In a preferred embodiment of the process, the agent (a) can now be rinsed from the keratinic materials before the agent (b) is applied to the hair in the subsequent step.

Stains with equally good wash fastnesses were obtained when agent (b) was applied to the keratinous materials that were still exposed to agent (a).

In step (4), agent (b) is now applied to the keratinous materials. After application, let the agent (b) act on the hair.

Even with a short contact time of the agent (b), the process allows the production of dyeing's with particularly good intensity and wash fastness. Contact times of 10 seconds to 10 minutes, preferably 20 seconds to 5 minutes and most preferably 30 seconds to 3 minutes on the keratinous materials, on human hair, have proven to be particularly advantageous.

In step (6), the agent (b) (and any agent (a) still present) is now rinsed out of the keratinous material with water.

Subsequently, the agent (c) is applied to the keratinous materials in a post-treatment step. Agent (c) is also left to act on the keratinous materials and then rinsed out again with water.

The positive effects achieved by agent (c) are particularly long-lasting if agent (c) is applied repeatedly—for example during regular hair washing.

In the context of a further form of execution, a procedure comprising the following steps in the order indicated is particularly preferred

(1) Application of the agent (a) on the keratinous material,
(2) Allow the agent (a) to act for a period of 10 seconds to 10 minutes, preferably from 10 seconds to 5 minutes,
(3) if necessary, rinse the keratinous material with water,
(4) Application of agent (b) on the keratinous material,
(5) Allowing the agent (b) to act for a period of 30 seconds to 30 minutes, preferably from 30 seconds to 10 minutes,
(6) Rinse the keratinous material with water,
(7) Application of the agent (c) on the keratinous material,
(8) allowing the agent (c) to act for a period of from 30 seconds to 10 minutes, preferably from 30 seconds to 50 minutes; and
(9) Rinse the keratinous material with water, wherein the sequence of steps (7), (8) and (9) is performed at least twice.

In the context of a further form of execution, a procedure comprising the following steps in the order indicated is particularly preferred

(1) Application of the agent (a) on the keratinous material,
(2) Allow the agent (a) to act for a period of 10 seconds to 10 minutes, preferably from 10 seconds to 5 minutes,
(3) if necessary, rinse the keratinous material with water,
(4) Application of agent (b) on the keratinous material,
(5) Allowing the agent (b) to act for a period of 30 seconds to 30 minutes, preferably from 30 seconds to 10 minutes,
(6) Rinse the keratinous material with water,
(7) Application of the agent (c) on the keratinous material,
(8) allowing the agent (c) to act for a period of from 30 seconds to 10 minutes, preferably from 30 seconds to 50 minutes; and
(9) Rinse the keratinous material with water,
(10) Application of the agent (c) on the keratinous material,
(11) allowing the agent (c) to act for a period of from 30 seconds to 10 minutes, preferably from 30 seconds to 50 minutes; and
(12) Rinse the keratinous material with water.

In this embodiment, the sequence of steps (1) to (9) takes place within a few hours. There may be a period of a few days between carrying out steps (9) and (10) to (12).

Multi-Component Packaging Unit (Kit-of-Parts)

In the process as contemplated herein, agents (a), (b) and (c) are applied to the keratinous materials, i.e., the three agents (a), (b) and (c) are respectively the ready-to-use agents.

To increase user comfort, the user is preferably provided with all required resources in the form of a multi-component packaging unit (kit-of-parts).

A second subject of the present disclosure is therefore a multi-component packaging unit (kit-of-parts) for coloring keratinous material, comprehensively packaged separately from one another
a first container comprising an agent (a), wherein the agent (a) comprises at least one organic silicon compound,
a second container comprising an agent (b), the agent (b) comprising at least one colorant compound including comprising selected from the group of pigments and/or direct dyes,
and a third container comprising an agent (c), wherein the agent comprises (c):
   (c1) a hydroxy terminated polyorganosiloxane, and
   (c2) a reaction product of a hydroxy terminated polyorganosiloxane with an acid and/or an alcohol and/or a wax.

The organic silicon compounds included in agent (a) of the kit correspond to the organic silicon compounds that were also used in agent (a) of the method described above.

The colorant compounds from the group of pigments and/or direct dyes included in agent (b) of the kit correspond to the colorant compounds from the group of pigments and/or direct dyes that were also used in agent (b) of the previously described process.

The hydroxy-terminated polyorganosiloxanes (c1) and reaction products of a hydroxy-terminated polyorganosiloxane with an acid and/or an alcohol and/or a wax (c2) included in agent (c) of the kit, which were also used in agent (c) of the method described above.

Agent (a) comprises, with the organic silicon compound(s), a class of reactive compounds capable of undergoing hydrolysis and/or oligomerization and/or polymerization in the presence of water as previously described. As a result of their high reactivity, these organic silicon compounds form a film on the keratinous material.

To avoid premature hydrolysis, oligomerization and/or polymerization, it may be preferable to prepare the ready-to-use agent (a) only shortly before use.

In the context of a further embodiment, a multi-component packaging unit (kit-of-parts) for coloring keratinic material is preferably packaged separately from one another
a first container comprising an agent (a1), wherein the agent (a1) comprises at least one organic silicon compound,
a second container comprising an agent (a2), wherein the agent (a2) comprises water,
a third container comprising an agent (b), the agent (b) comprising at least one colorant compound including comprising selected from the group of pigments and/or direct dyes,
and a fourth container comprising an agent (c), wherein the agent comprises (c):
   (c1) a hydroxy terminated polyorganosiloxane, and
   (c2) a reaction product of a hydroxy terminated polyorganosiloxane with an acid and/or an alcohol and/or a wax.

To provide a formulation that is as stable as possible during storage, the agent (a1) itself is preferably packaged with low or no water.

In a preferred embodiment, a multicomponent packaging unit (kit-of-parts) is wherein the agent (a1)—based on the total weight of the agent (a1)—has a water content of less than 10% by weight, preferably of less than 5% by weight, more preferably of less than 1% by weight, even more preferably of less than 0.1% by weight and very particularly preferably of less than 0.01% by weight.

The agent (a2) comprises water. In a preferred embodiment, a multicomponent packaging unit (kit-of-parts) is wherein the agent (a2)—based on the total weight of the agent (a2)—has a water content of from 15 to 100% by weight, preferably from 35 to 100% by weight, more preferably from 55 to 100% by weight, still more preferably from 65 to 100% by weight and very particularly preferably from 75 to 100% by weight.

Within this version, the ready-to-use agent (a) is now produced by mixing agents (a1) and (a2).

For example, the user can first mix or shake the agent (a1) comprising the organic silicon compound(s) with the water-comprising agent (a2). The user can now apply this mixture of (a1) and (a2)—either directly after its preparation or after a short reaction time of 10 seconds to 20 minutes—to the keratinous materials. Following this, the user can apply means (b) and (c) as previously described.

Regarding the further preferred embodiments of the multicomponent packaging unit, the same applies mutatis mutantis as to the process.

### Examples

### 1. Formulations

The following formulations have been produced (unless otherwise indicated, all figures are in % by weight)

| Pretreatment agent, agent (a) | |
|---|---|
| (3-Aminopropyl)triethoxysilane | 2.0 |
| Methyltrimethoxysilane | 7.0 |
| Ammonia/citric acid | ad pH 9.5 |
| Water | ad 100 |

The silanes were mixed with a portion of water, this mixture was left for 30 minutes. Then the pH value was adjusted to the desired value by adding citric acid/ammonia. Water was then added to make up to 100 g.

| Dye, agent (b) | | | | |
|---|---|---|---|---|
| Colorona Bronze (Merck, | 1.0 | | | |
| Glimmer, CI 77491, | | | | |
| Eisenoxide, CI77019) | | | | |
| Unipure Red LC 3071 | 1.0 | | | |
| (Sensient, Aluminum | | | | |
| hydroxide, CI 15850) | | | | |
| PVP K 30 (Ashland, ISP, | 4.5 | | | |
| Polyvinylpyrrolidone) | | | | |
| Dermacryl 79 (Akzo Nobel, | 4.5 | | | |
| Acrylates/Octylacrylamide | | | | |
| Copolymer, CAS-Nr. | | | | |
| 129702-02-9) | | | | |
| Ammonia (25% aqueous | ad pH 10 | | | |
| solution) | | | | |
| Water | ad 100 | | | |
| After-treatment agent, | | | | |
| agent (c) | (cI) | (cII) | (cIII) | (CIV) |
| Belsil ® ADM 8301 E* | 5 | 5 | 5 | 5 |
| Fancorsil LEM-2** | 0.5 | 0.5 | — | — |
| SilSense ® IWS Silicone*** | — | — | 0.5 | 0.5 |
| Lactic acid | 0.6 | 0.6 | 0.6 | 0.6 |
| Cetearyl Alcohol (INCI) | — | 5 | — | 5 |
| Ceteareth-20 (INCI) | — | 1 | — | 1 |
| Water | ad 100 | ad 100 | ad 100 | ad 100 |

*Active silicone content: 27-31 wt..-%
**Active silicone content: 100 wt.-%
***Active silicone content: 100 wt.-%

### 2. Application

One strand of hair (Kerling, Euronatural hair white) was dipped into the medium (a) and left in it for 1 minute. Afterwards, excess product (a) was stripped from each strand of hair. Each strand of hair was briefly washed with water. Excess water was scraped off each strand of hair.

Subsequently, the hair strands were each dipped in the agent (b) and left in it for 1 minute. Afterwards, excess agent (b) was stripped from each strand of hair. Each strand of hair was briefly washed with water. Excess water was scraped off each strand of hair.

Then, the hair strands were each wetted with a small amount of the agent (c). The agent (c) was left to act for 1 minute. Then washed out with water and dried the strand of hair. Subsequently, the strands were visually evaluated.

| Examples | 1 | 2 | 3 | 4 |
|---|---|---|---|---|
| Agent (a) | (a) | (a) | (a) | (a) |
| Agent (b) | (b) | (b) | (b) | (b) |
| Agent (c) | (cI) | (cII) | (cIII) | (CIV) |
| Coloring | bronze | bronze | bronze | bronze |
| | red +++ | red ++ | red +++ | red +++ |

Color intensity: + = bad ++ = average +++ = excellent

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A process for dyeing keratinous material, in particular human hair, comprising the following steps:
Application of an agent (a) to the keratinous material, wherein the agent (a) comprises at least one organic silicon compound,
Application of an agent (b) to the keratinous material, the agent (b) comprising at least one colorant compound selected from the group consisting of pigments and/or direct dyes, and
Application of an agent (c) to the keratinous material, wherein the agent (c) comprises:
(c1) a hydroxy-terminated polyorganosiloxane, and
(c2) a reaction product of a hydroxy terminated polyorganosiloxane with an acid and/or an alcohol and/or a wax.

2. The process according to claim 1, characterized in that the agent (a) contains at least one organic silicon compound selected from silanes having one, two or three silicon atoms, the organic silicon compound preferably comprising one or more basic chemical functions and one or more hydroxyl groups or hydrolysable groups per molecule.

3. The process according to claim 1, characterized in that the agent (a) comprises at least one organic silicon compound of the formula (I) and/or (II)
R1R2N-L-Si(OR3)a(R4)b (I),
where
R1, R2 independently represent a hydrogen atom or a C1-C6 alkyl group,
L is a linear or branched divalent C1-C20 alkylene group,
R3 is a hydrogen atom or a C1-C6 alkyl group,
R4 stands for a C1-C6 alkyl group,
a, stands for an integer from 1 to 3, and
b is the integer 3-a, and
wherein in the organic silicon compound of formula (II)
(R5O)c(R6)dSi-(A)e-[NR7-(A′)]f-[O-(A″)]g-[NR8-(A′″)]h-Si(R6′)d′(OR5′)c′ (II),
R5, R5′, R5″ independently represent a hydrogen atom or a C1-C6 alkyl group,
R6, R6′ and R6″ independently represent a C1-C6 alkyl group,
A, A′, A″, A′″ and A″″ independently represent a linear or branched divalent C1-C20 alkylene group,
R7 and R8 independently represent a hydrogen atom, a C1-C6 alkyl group, a hydroxy C1-C6 alkyl group, a C2-C6 alkenyl group, an amino C1-C6 alkyl group or a group of formula (III),
-(A″″)-Si(R6″)d″(OR5″)c″ (III),
c″ stands for an integer from 1 to 3,
d stands for the integer 3-c,
c″ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the residues from e, f, g and h is different from 0.

4. The process according to claim 1, characterized in that the agent (a) comprises at least one organic silicon compound of formula (I),
R1R2N-L-Si(OR3)a(R4)b (I),
where
R1, R2 both represent a hydrogen atom, and
L represents a linear, divalent C1-C6-alkylene group, preferably a propylene group (—CH2-CH2-CH2-) or an ethylene group (—CH2-CH2-),
R3 represents a hydrogen atom, an ethyl group, or a methyl group,
R4 represents a methyl group or an ethyl group,
a stands for the number 3 and
b stands for the number 0.

5. The process according to claim 1, characterized in that the agent (a) comprises at least one organic silicon compound of formula (I) selected from the group consisting of
(3-Aminopropyl)triethoxysilane
(3-Aminopropyl)trimethoxysilane
1-(3-Aminopropyl)silantriol
(2-Aminoethyl)triethoxysilane
(2-Aminoethyl)trimethoxysilane
1-(2-Aminoethyl)silantriol
(3-Dimethylaminopropyl)triethoxysilane
(3-Dimethylaminopropyl)trimethoxysilane
1-(3-Dimethylaminopropyl)silantriol
(2-Dimethylaminoethyl)triethoxysilane.
(2-Dimethylaminoethyl)trimethoxysilane,
1-(2-Dimethylaminoethyl)silantriol and
Mixtures of these.

6. The process according to any one of claims 1 to 5, characterized in that the agent (a) comprises at least one organic silicon compound of the formula (II),
(R5O)c(R6)dSi-(A)e-[NR7-(A′)]f-[O-(A″)]g-[NR8-(A′″)]h-Si(R6′)d′(OR5′)c′ (II),
where
e and f both stand for the number 1,
g and h both stand for the number 0,
A and A′ independently represent a linear, divalent C1-C6 alkylene and
R7 represents a hydrogen atom, a methyl group, a 2-hydroxyethyl group, a 2-alkenyl group, a 2-aminoethyl group or a group of formula (III).

7. The process according to claim 1, characterized in that it comprises (a) at least one organic silicon compound of the formula (II) which is selected from the group consisting of
3-(Trimethoxysilyl)-N-[3-(trimethoxysilyl) propyl]-1-propanamine
3-(Triethoxysilyl)-N-[3-(triethoxysilyl) propyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl)propyl]-1-propane amine
2-[Bis[3-(trimethoxysilyl) propyl]amino]-ethanol
2-[Bis[3-(triethoxysilyl) propyl]amino]-ethanol
3-(Trimethoxysilyl)-N,N-bis[3-(trimethoxysilyl) propyl]-1-propanamine
3-(Triethoxysilyl)-N,N-bis[3-(triethoxysilyl) propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine,
N,N-bis[3-(triethoxysilyl)propyl]-2-propen-1-amine and
Mixtures of these.

8. The process according to claim 1, characterized in that the agent (a) comprises at least one organic silicon compound of formula (IV).
R9Si(OR10)k(R11)m (IV),
where
R9 stands for a C1-C18 alkyl group,
R10 represents a hydrogen atom or a C1-C6 alkyl group,
R11 represents a C1-C6 alkyl group
k is an integer from 1 to 3, and
m stands for the integer 3-k.

9. The process according to claim 1, characterized in that the agent (a) comprises at least one organic silicon compound of formula (IV) selected from the group consisting of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Octyldecyltrimethoxysilane,
Octyldecyltriethoxysilane and
Mixtures of these.

10. The process according to claim 1, characterized in that the agent (c) is, as hydroxy terminated polyorganosiloxane, a polyorganosiloxane of formula (I) 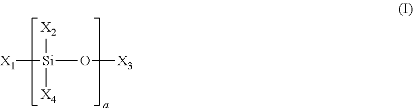
Includes,
wherein
X1 and X2 are independently OH, OR1, R2, O-PDMS or O-fSiloxane,
X3 is hydrogen or a monovalent hydrocarbon radical having 1 to 8 carbon atoms per radical,
PDMS or fSiloxane,
X4 is a remainder of the formula 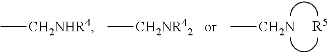
and
a is a number from 1 to 100,
where
R1 is an alkyl radical having 1 to 8 carbon atoms,
R2 is a monovalent, saturated, or unsaturated hydrocarbon radical which is optionally substituted by the elements N, P, S, O, Si, and halogen and has 1 to 200 carbon atoms per radical,
PDMS stands 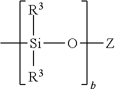
for,
fSiloxan stands 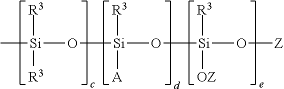
for,
R3 independent of one another is in each case a monovalent saturated or unsaturated hydrocarbon radical having 1 to 200 carbon atoms per radical and optionally substituted by the elements N, P, S, O, Si, and halogen,
A is a radical of the formula R6-[NR7-R8-]fNR72,
where
R6 is a divalent linear or branched hydrocarbon radical containing 3 to 18 carbon atoms,
R7 is a hydrogen atom, an alkyl radical having 1 to 8 carbon atoms or an acyl radical,
R8 is a divalent hydrocarbon radical containing 1 to 6 carbon atoms,
b is a number from 1 to 2000,
c is 0 or a number from 1 to 2000,
d is a number from 1 to 1000,
e is 0 or a number from 1 to 5,
f is 0, 1, 2, 3 or 4,
Z is hydrogen, an alkyl radical having from 1 to 8 carbon atoms 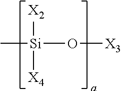
or,
R4 is a monovalent hydrocarbon radical optionally containing N and/or O atoms and having 1 to 18 carbon atoms, and
R5 is a divalent hydrocarbon radical optionally containing N and/or O atoms and having 3 to 12 carbon atoms,
with the proviso that the polyorganosiloxane of the formula (I) has at least one terminal OH group.

11. The process according to claim 1, characterized in that the polyorganosiloxane of formula (I) contains a morpholinomethyl radical as radical X4.

12. The process according to claim 1, characterized in that the agent (c) comprises, as hydroxy terminated polyorganosiloxane (c1), at least one compound known under the INCI designation amodimethicone/morpholinomethyl silsesquioxane copolymer.

13. The process of claim 1, characterized in that the agent (c) comprises a reaction product of a hydroxy-terminated polyorganosiloxane with an acid selected from the group consisting of reaction products of a hydroxy-terminated polyorganosiloxane with a fatty acid, reaction products of a hydroxy-terminated polyorganosiloxane with an amino acid, reaction products of a hydroxy-terminated polyorganosiloxane with an α-hydroxy acid, and mixtures thereof.

14. The process of claim 13, characterized in that the agent (c) comprises a reaction product of a hydroxy terminated polyorganosiloxane with a fatty acid selected from the group consisting of reaction products of dimethiconol with fatty acids derived from meadowfoam seed oil (INCI: dimethiconol meadowfoamate), reaction products of dimethiconol with stearic acid (INCI: dimethiconol stearate) and mixtures thereof.

15. (canceled)
